SEQUENCE LISTING (2 sequences)

SEQ ID NO: 1 - protein, 61 aa, synthetic construct
SHLVKCAEKEKTFCVNGGECFMVKDLSNPSRYLCKCPNEFTGDRCQNYVMASFYKAEELYQ

SEQ ID NO: 2 - protein, 23 aa, synthetic construct
AEKEKTFCVNGGECFMVKDLSNP

(19) United States
(12) Patent Application Publication
(10) Pub. No.: US 2026/0103496 A1
(43) Pub. Date: Apr. 16, 2026

(54) NEUREGULIN AND USE THEREOF

(71) Applicant: ZENSUN (SHANGHAI) SCIENCE & TECHNOLOGY, CO., LTD., Shanghai (CN)

(72) Inventor: Mingdong ZHOU, Shanghai (CN)

(21) Appl. No.: 19/116,520

(22) PCT Filed: Sep. 28, 2023
(86) PCT No.: PCT/CN2023/122655

§ 371 Date: Mar. 28, 2025

(30) Foreign Application Priority Data
Sep. 30, 2022 (CN) 202211231741.1

(51) Int. Cl. C07K 14/485; A61K 38/00; A61P 9/10

(52) U.S. Cl. CPC C07K 14/485; A61P 9/10; A61K 38/00

(57) ABSTRACT

Provided is use of a neuregulin or a functional fragment thereof, or a nucleic acid encoding a neuregulin or a functional fragment thereof, or a substance for improving the yield and/or function of a neuregulin in a human body in the preparation of a medicament for preventing, treating or delaying the onset of heart failure in a heart failure patient. The medicament can improve the cardiac function of the heart failure patient and reverse ventricular remodeling. Also provided are a method and device for preventing and treating a heart failure patient or delaying the onset of heart failure in a heart failure patient.

### TECHNICAL FIELD

The present invention relates to the field of medicine. Specifically, the present invention relates to the use of neuregulin in the preparation of a drug for preventing, treating or alleviating human heart failure, and to methods for preventing, treating, or alleviating human heart failure using the neuregulin.

### SEQUENCE LISTING

The present application is being filed along with a Sequence Listing in electronic format.

The Sequence Listing is provided as a file entitled Sequence Listing.xml created on Mar. 27, 2025, which is 2.67 KB in size. The information in the electronic format of the Sequence Listing is incorporated herein by reference in its entirety.

### BACKGROUND

Heart failure (HF) is a syndrome characterized by cardiac dysfunction resulting from various heart diseases. Current pharmacological treatments for heart failure primarily focus on angiotensin-converting enzyme (ACE) inhibitors, which act as vasodilators to dilate blood vessels, reduce blood pressure, and alleviate cardiac load. While the therapy by ACE inhibitors demonstrates statistically significant mortality reduction, there is only 3%-4% average reduction in the actual mortality, accompanied by potential side effects. Other therapeutic options for preventing or treating heart failure also present limitations. For instance, heart transplantation is substantially more expensive and invasive compared with a drug therapy, with additional constraints imposed by donor organ availability. Mechanical devices, such as dual-chamber pacemakers, similarly are also invasive and of high cost. Therefore, there is a need for novel therapeutic approaches due to the inadequacies of existing treatment.

A promising new treatment involves the administration of neuregulin (NRG; Heregulin, HRG), also known as glial growth factor (GGF) and new differentiation factor (NDF) to patients with heart failure or patients at risk of heart failure. These glycoproteins, the molecular weight of which is about 44 KD, transmit signals between cells and are ligands of tyrosine kinase receptor ErbB family. Neuregulin family contains four members: NRG1, NRG2, NRG3 and NRG4. NRGs involves a series of biological reactions: stimulating the differentiation of breast cancer cells and secreting milk protein; inducing neural crest cells to differentiate into Schwann cells; stimulating skeletal muscle cells to synthesize acetylcholine receptors; and promoting the survival of myocardial cells and DNA synthesis. In vivo studies with homozygous mouse embryos with serious defects in neuregulin gene proved that neuregulin is necessary for heart and nerve development. NRG1 plays an important role in nervous system, heart and breast, and there is evidence that NRG1 signaling plays a role in the development and function of other organ systems and the pathogenesis of human diseases, including schizophrenia and breast cancer. There are many isomers of NRG1. Studies on mutant mice (knockout mice) show that different isomers in N-terminal region or similar region of epidermal growth factor (EGF) have different functions in vivo.

There have been various studies on the function of neuregulin, however, more researches are needed on how to use neuregulin and how to achieve better therapeutic effects in some sub-populations.

Additionally, left ventricular (LV) function assessment plays a crucial role in clinical decision-making for patients with cardiovascular diseases. Specifically, the left ventricular end-systolic volume index (LVESVI) serves as a metric for evaluating left ventricular function, and the increase in LVESVI correlates with increased incidence of adverse outcomes including mortality and hospitalization. Currently, heart failure patients exhibiting particularly high LVESVI values (e.g., exceeding 135 mL/m²) demonstrate significantly poorer prognoses, while concrete treatment protocols for this specific patient population remain absent in literatures. The management of heart failure patients with markedly elevated LVESVI values continues to represent both an unmet clinical need and a substantial medical challenge.

### SUMMARY OF THE INVENTION

The purpose of the present invention is to provide a method for improving cardiac function in patients with heart failure by using neuregulin, comprising using a drug containing Neuregulin in a specific population of patients suffering from chronic heart failure.

In the first aspect, the present invention provides the use of a neuregulin or a functional fragment thereof, or a nucleic acid encoding the neuregulin or a functional fragment thereof, or a substance that enhances the production and/or function of the neuregulin in the human body, in the preparation of a drug for preventing, treating a patient with heart failure, or delaying the onset of heart failure in a patient, and the drug can improve cardiac function in a patient with heart failure.

The present invention provides the use of the neuregulin in the preparation of a drug for preventing, treating a patient with heart failure, delaying the onset of heart failure in a patient, or improving cardiac function in a patient with heart failure.

In some embodiments, the patient with heart failure suffers from severe heart failure as assessed by left ventricular function index. In some embodiments, for example, the LVESVI value of the patient with severe heart failure is at least 3 times that of a healthy individual. In some embodiments, for example, the LVESVI value of the patient with severe heart failure is at least 4 times that of a healthy individual. In some embodiments, for example, the LVESVI value of the patient with severe heart failure exceeds that of a healthy individual by 100 mL/m². In some embodiments, for example, the patient with severe heart failure has LVESVI values within approximately the top 40% highest among all heart failure patients. In some embodiments, for example, the patient with severe heart failure has LVESVI values greater than or equal to approximately 100, 105, 110, 115, 120, 125, 130, 135, 140, 145, 150, 155, 160, 165, or 170 mL/m². In some embodiments, for example, the patient with severe heart failure has LVESVI values greater than or equal to approximately 135 mL/m². In some embodiments, the patient with severe heart failure as said above represents a population with severe ventricular remodeling. In some embodiments, the patient with severe heart failure as said above represents a population with enlarged hearts.

In some embodiments, for example, the present invention is used for preventing, treating, or delaying a patient with heart failure with an LVESVI greater than or equal to about 100, 105, 110, 115, 120, 125, 130, 135, 140, 145, 150, 155, 160, 165, or 170 mL/m². In some embodiments, for example, the patient with heart failure has an LVESVI value of not less than about 100, 105, 110, 115, 120, 125, 130, 135, 140, 145, 150, 155, 160, 165, or 170 mL/m².

In some embodiments, the patient with heart failure has severe ventricular remodeling.

In some embodiments, the patient with heart failure has an LVESVI of ≥100 mL/m², ≥110 mL/m², ≥120 mL/m², ≥130 mL/m², ≥135 mL/m², ≥140 mL/m², ≥145 mL/m², ≥150 mL/m², ≥155 mL/m², ≥160 mL/m², ≥165 mL/m², or ≥170 mL/m² at baseline before administration; preferably ≤135 mL/m²; or
the patient with heart failure has an LVESVI of 100-350 mL/m², 110-350 mL/m², 120-350 mL/m², 130-350 mL/m², or 135-350 mL/m² at baseline before administration; preferably 135-350 mL/m².

In a preferred embodiment, the patient with heart failure is a patient with chronic heart failure.

In a preferred embodiment, the patient with heart failure is classified as NYHA II or III according to the New York Heart Association (NYHA) functional classification, or has a plasma NT-proBNP level not exceeding 4000 pg/ml.

In a preferred embodiment, in the patient with heart failure, a male patient has a plasma NT-proBNP level not exceeding 1700 pg/ml, and a female patient has a plasma NT-proBNP level not exceeding 4000 pg/ml.

In a preferred embodiment, “improving cardiac function in the patient with heart failure” refers to improving LVESVI and/or LVEDVI.

In a specific embodiment, the LVESVI of the patient with heart failure is reduced by an amount equal to or greater than 10 mL/m²; preferably equal to or greater than 20 mL/m².

In a specific embodiment, the LVEDVI of the patient with heart failure is reduced by an amount equal to or greater than 10 mL/m²; preferably equal to or greater than 20 mL/m².

In a specific embodiment, “improving cardiac function in the patient with heart failure” refers to simultaneously improving both LVESVI and LVEDVI in an individual patient with heart failure.

In a specific embodiment, both LVESVI and LVEDVI are simultaneously reduced by an amount equal to or greater than 20 or 25 mL/m² in an individual patient with heart failure.

In a specific embodiment, the patient with heart failure experience reversal of ventricular remodeling.

In a preferred embodiment, the improvement in LVESVI and/or LVEDVI is achieved after 10 days of the administration of neuregulin.

In some embodiments, the neuregulin comprises neuregulin 1 (NRG-1), neuregulin 2 (NRG-2), neuregulin 3 (NRG-3), and neuregulin 4 (NRG-4).

In some embodiments, the neuregulin comprises neuregulin 1 (NRG-1).

In some embodiments, the neuregulin comprises the EGF-like domain of neuregulin 1 (NRG-1).

In some embodiments, the neuregulin is a protein, the amino acid sequence of which is shown in SEQ ID NO:1.

In the second aspect, the present invention provides a method for preventing, treating a patient with heart failure, or delaying the onset of heart failure in a patient, comprising administering to a patient in need thereof an effective amount of neuregulin or a functional fragment thereof, or a nucleic acid encoding the neuregulin or a functional fragment thereof, or a substance that enhances the production and/or function of the neuregulin in the human body, and the method can improve cardiac function in the patients with heart failure.

The present invention provides a method for preventing, treating a patient with heart failure, delaying the onset of heart failure in a patient, or improving cardiac function in a patient with heart failure, comprising administering to a patient in need thereof an effective amount of neuregulin.

In some embodiments, the patient with heart failure suffers from severe heart failure as assessed by left ventricular function index. In some embodiments, for example, the LVESVI value of the patient with severe heart failure is at least 3 times that of a healthy individual. In some embodiments, for example, the LVESVI value of the patient with severe heart failure is at least 4 times that of a healthy individual. In some embodiments, for example, the LVESVI value of the patient with severe heart failure exceeds that of a healthy individual by 100 mL/m². In some embodiments, for example, the patient with severe heart failure has LVESVI values within approximately the top 40% highest among all heart failure patients. In some embodiments, for example, the patient with severe heart failure has LVESVI values greater than or equal to approximately 100, 105, 110, 115, 120, 125, 130, 135, 140, 145, 150, 155, 160, 165, or 170 mL/m². In some embodiments, for example, the patient with severe heart failure has LVESVI values greater than or equal to approximately 135 mL/m². In some embodiments, the patient with severe heart failure as said above represents a population with severe ventricular remodeling. In some embodiments, the patient with severe heart failure as said above represents a population with enlarged hearts.

In some embodiments, for example, the present invention is used for preventing, treating, or delaying a patient with heart failure with an LVESVI greater than or equal to about 100, 105, 110, 115, 120, 125, 130, 135, 140, 145, 150, 155, 160, 165, or 170 mL/m². In some embodiments, for example, the patient with heart failure has an LVESVI value of not less than about 100, 105, 110, 115, 120, 125, 130, 135, 140, 145, 150, 155, 160, 165, or 170 mL/m².

In some embodiments, the patient with heart failure has severe ventricular remodeling.

In some embodiments, the patient with heart failure has an LVESVI of ≥100 mL/m², ≥110 mL/m², ≥120 mL/m², ≥130 mL/m², ≥135 mL/m², ≥140 mL/m², ≥145 mL/m², ≥150 mL/m², ≥155 mL/m², ≥160 mL/m², ≥165 mL/m², or ≥170 mL/m² at baseline before administration; preferably ≤135 mL/m²; or the patient with heart failure has an LVESVI of 100-350 mL/m², 110-350 mL/m², 120-350 mL/m², 130-350 mL/m², or 135-350 mL/m² at baseline before administration; preferably 135-350 mL/m².

In a preferred embodiment, the patient with heart failure is a patient with chronic heart failure.

In a preferred embodiment, the patient with heart failure is classified as NYHA II or III according to the New York Heart Association (NYHA) functional classification, or has plasma NT-proBNP levels not exceeding 4000 pg/ml.

In a preferred embodiment, in the patient with heart failure, a male patient has plasma NT-proBNP levels not exceeding 1700 pg/ml, and a female patient has plasma NT-proBNP levels not exceeding 4000 pg/ml.

In a preferred embodiment, “improving cardiac function in the patient with heart failure” refers to improving LVESVI and/or LVEDVI.

In a specific embodiment, the LVESVI of the patient with heart failure is reduced by an amount equal to or greater than 10 mL/m²; preferably equal to or greater than 20 mL/m².

In a specific embodiment, the LVEDVI of the patient with heart failure is reduced by an amount equal to or greater than 10 mL/m²; preferably equal to or greater than 30 mL/m².

In a specific embodiment, “improving cardiac function in the patient with heart failure” refers to simultaneously improving both LVESVI and LVEDVI in an individual patient with heart failure.

In a specific embodiment, both LVESVI and LVEDVI are simultaneously reduced by an amount equal to or greater than 20 or 25 mL/m² in an individual patient with heart failure.

In a specific embodiment, the patient with heart failure experience reversal of ventricular remodeling.

In a preferred embodiment, the improvement in LVESVI and/or LVEDVI is achieved after 10 days of the administration of neuregulin.

In a preferred embodiment, the method comprises administering neuregulin to a patient via an introduction regimen.

In a preferred embodiment, the introduction regimen comprises continuously administering the drug component for at least 3, 5, 7, or 10 consecutive days.

In a preferred embodiment, the method comprises administering a maintenance regimen to the patient for at least 3, 6, or 12 months.

In a preferred embodiment, the maintenance regimen comprises administering the drug every 3, 5, 7, or 10 days.

In a preferred embodiment, the method further comprises a step of assessing whether the patient in need of preventing, treating or delaying heart failure is suitable for neuregulin therapy prior to administering the treatment to the patient.

In a preferred embodiment, the step of assessing whether the patient is suitable for neuregulin therapy comprises performing a NYHA cardiac function classification assessment on the patient with heart failure.

In a preferred embodiment, a patient with NYHA Class II or NYHA Class III according to NYHA cardiac function classification is suitable for neuregulin therapy.

In a preferred embodiment, the step of assessing whether the patient is suitable for neuregulin therapy further comprises measuring plasma NT-proBNP in the patient with heart failure.

In a preferred embodiment, the plasma NT-proBNP level does not exceed 4000 pg/ml.

In a preferred embodiment, a male patient with heart failure has a plasma NT-proBNP level not exceeding 1700 pg/ml, and a female patient has a plasma NT-proBNP level not exceeding 4000 pg/ml.

In some embodiments, the neuregulin comprises neuregulin 1 (NRG-1), neuregulin 2 (NRG-2), neuregulin 3 (NRG-3), and neuregulin 4 (NRG-4).

In some embodiments, the neuregulin comprises neuregulin 1 (NRG-1).

In some embodiments, the neuregulin comprises the EGF-like domain of neuregulin 1 (NRG-1).

In some embodiments, the neuregulin is a protein, the amino acid sequence of which is shown in SEQ ID NO:1.

In the third aspect, the present invention provides a device for preventing, treating a patient with heart failure, or delaying the onset of heart failure in a patient, wherein the device is a device for improving LVESVI and/or LVEDVI in the patient with heart failure, comprising:
a treatment module that administers neuregulin therapy to a patient suitable for neuregulin therapy based on whether:
the NYHA cardiac functional classification of the patient is NYHA class II or III,
the plasma NT-proBNP of the patient does not exceed 4000 pg/ml, or,
the baseline LVESVI of the patient prior to the administration is ≥135 mL/m².

In a preferred embodiment, the device further comprises:
a detection module configured to perform NYHA cardiac function classification on the patient, measure plasma NT-proBNP level in the patient with heart failure, or determine the baseline LVESVI value of the patient before the administration; and
an analysis and judgment module configured to analyze and determine whether:
the NYHA cardiac function classification of the patient is NYHA Class II or NYHA Class III, or
the plasma NT-proBNP level of the patient does not exceed 4000 pg/ml, or
the baseline LVESVI of the patient before the administration is ≥135 mL/m².

In a preferred embodiment, a male patient with heart failure has a plasma NT-proBNP level not exceeding 1700 pg/ml, and a female patient has a plasma NT-proBNP level not exceeding 4000 pg/ml.

In a preferred embodiment, the neuregulin is a protein, the amino acid sequence of which is shown in SEQ ID NO:1.

In a preferred embodiment, the patient with heart failure represents a population with severe ventricular remodeling.

In a preferred embodiment, the patient with heart failure is a patient with chronic heart failure.

In a preferred embodiment, the improvement in LVESVI and/or LVEDVI in the patient with heart failure refers to a reduction in LVESVI and/or LVEDVI.

In a preferred embodiment, the LVESVI in the patient with heart failure is reduced by 310 mL/m²; preferably ≥20 mL/m².

In a preferred embodiment, the LVEDVI in the patient with heart failure is reduced by 210 mL/m²; preferably ≥20 mL/m².

In a preferred embodiment, the patient with heart failure has a baseline LVESVI ≥135 mL/m² prior to the administration.

In a preferred embodiment, the patient with heart failure is a patient with heart failure whose LVESVI and LVEDVI are simultaneously improved.

In a preferred embodiment, both LVESVI and LVEDVI in the patient with heart failure are simultaneously reduced by an amount equal to or greater than 20 or 25 mL/m².

In a preferred embodiment, the patient with heart failure experience reversal of ventricular remodeling.

In a preferred embodiment, the improvement in LVESVI and/or LVEDVI is achieved after 10 days of the administration of neuregulin.

In a preferred embodiment, the treatment module comprises a means for administering neuregulin to a patient via an introduction regimen.

In a preferred embodiment, the treatment module comprises a means for continuously administering the neuregulin for at least 3, 5, 7, or 10 consecutive days.

In a preferred embodiment, the method comprises a means for administering a maintenance regimen to the patient for at least 3, 6, or 12 months.

In a preferred embodiment, the maintenance regimen comprises administering the drug every 3, 5, 7, or 10 days.

In some embodiments, the neuregulin comprises neuregulin 1 (NRG-1), neuregulin 2 (NRG-2), neuregulin 3 (NRG-3), and neuregulin 4 (NRG-4).

In some embodiments, the neuregulin comprises neuregulin 1 (NRG-1).

In some embodiments, the neuregulin comprises the EGF-like domain of neuregulin 1 (NRG-1).

In some embodiments, the neuregulin is a protein, the amino acid sequence of which is shown in SEQ ID NO:1.

It should be understood that, within the scope of the present invention, the above technical features of the present invention and the technical features specifically described in the following (such as in the Examples) can be combined with each other to form new or preferred technical solutions. Such technical solutions will not be described one by one due to the limited contents.

### DESCRIPTION OF DRAWINGS

FIG. 1 shows the percentage of patients in the Neuregulin group showing decreased or increased LVESVI at Day 30;

FIG. 2 shows the difference in LVESVI improvement from baseline at Day 30 between the Neuregulin and placebo groups;

FIG. 3 shows the difference in LVESVI worsening from baseline at Day 30 between the Neuregulin and placebo groups;

FIG. 4 shows the improvement in cardiac function index, LVESVI from baseline at Days 30 and 90;

FIG. 5 shows the difference in LVEDVI improvement from baseline at Day 30 between the Neuregulin and placebo groups;

FIG. 6 shows the difference in the worsening of LVEDVI from baseline at Day 30 between Neuregulin and placebo groups;

FIG. 7 shows significant improvements in both LVESVI and LVEDVI of an individual subject at Day 30;

FIG. 8 shows that Neuregulin resulted in a substantial LVESVI improvement (reduction by ≥13 mL/m²) within the enriched subgroup population (baseline LVESVI 2135 mL/m²) compared with the placebo group;

FIG. 9 shows that Neuregulin resulted in substantial improvements in both LVESVI and LVEDVI (both reduced by ≥20 mL/m²) in the enriched population (baseline LVESVI 2135 mL/m²) compared with the placebo group;

FIG. 10 shows that the response rate for substantial improvements in both LVESVI and LVEDVI (both reduced by ≥20 mL/m²) with Neuregulin in the enriched population (baseline LVESVI ≥135 mL/m²) was higher than that in the overall population;

FIG. 11a shows that at Day 30 after the administration, Neuregulin demonstrated a greater average improvement in LVESVI from baseline in the enriched population (baseline LVESVI ≥135 mL/m²) compared with the overall population;

FIG. 11b shows that at Day 30 after the administration, Neuregulin demonstrated a greater average improvement in LVEDVI from baseline in the enriched population (baseline LVESVI ≥135 mL/m²) compared with the overall population.

### MODES FOR CARRYING OUT THE INVENTION

The present invention comprises the following: significant therapeutic effects can be achieved for a patient with heart failure selected through New York Heart Association (NYHA) function classification and/or detection of NT-proBNP levels in the plasma through the neuregulin therapy.

The neuregulin described in this invention can improve cardiac function or prevent the deterioration of cardiac function in a patient with heart failure, with such improvement including reductions in LVESVI and/or LVEDVI. In a specific embodiment, significant concurrent reductions in both LVESVI and LVEDVI were observed in certain individual subjects. In other embodiments, the higher the baseline LVESVI level in an individual subject, the greater the improvement in cardiac function.

Unless otherwise specified, all technical and scientific terms used herein shall have the same meanings as commonly understood by a skilled person in the field to which this invention belongs. All patent documents, patent application documents, published patent literature, and other publications referenced herein are incorporated by reference. Where the definitions set forth in this section conflict with or contradict those described in the references as said above, the definitions provided in this section shall prevail.

Unless clearly indicated in the context, the singular forms “a,” “an,” and “the” used in this specification and the appended claims shall be construed to include plural references, and are interchangeable with “at least one” or “one or more.”

The present invention is based on neuregulin 10 (NRG10), a transmembrane protein. Its extracellular portion constitutes the N-terminal region, containing an immunoglobulin-like domain (Ig-like domain) and an epidermal growth factor-like domain (EGF-like domain), while the intracellular portion is the C-terminal region. Under the action of metalloproteinases in the extracellular matrix, the extracellular portion of neuregulin is enzymatically cleaved and present in a free state, thereby facilitating binding to ErbB receptors on the surface of neighboring cells, and thus activating corresponding cellular signaling pathways.

The ErbB receptor family is also categorized into four classes: ErbB1, ErbB2, ErbB3, and ErbB4, all of which are transmembrane proteins with molecular weights around 180-185 kDa. Except for ErbB2, all members contain a ligand-binding domain at the extracellular N-terminus. Similarly, except for ErbB3, all members possess tyrosine kinase activity at the intracellular C-terminus. Among them, ErbB1 serves as the receptor for epidermal growth factor, while both ErbB3 and ErbB4 function as receptors for neuregulin. Among the neuregulin receptors, only ErbB2 and ErbB4 exhibit relatively high expression levels in the heart.

When neuregulin binds to the extracellular domains of ErbB3 or ErbB4, it induces the formation of heterodimers between ErbB3, ErbB4 and other ErbB receptors (often including ErbB2), or homodimerization of ErbB4 itself, which subsequently leads to phosphorylation of the intracellular domains of these receptors. The phosphorylated intracellular domains can then interact with various intracellular signaling proteins, thereby activating downstream ERK or AKT signaling pathways and triggering a series of cellular responses, including stimulation or inhibition of cell proliferation, apoptosis, cell migration, cell differentiation, and cell adhesion.

Existing studies have demonstrated that the EGF-like domain of NRG-1, comprising approximately 50 to 64 amino acids, is sufficient for binding and activating these receptors. Previous researches have shown that neuregulin-1β (NRG-1β) can directly bind to ErbB3 and ErbB4 with high affinity. The orphan receptor ErbB2 can form heterodimers with either ErbB3 or ErbB4, which exhibits stronger binding affinity compared with homodimers of ErbB3 or ErbB4. Findings from neurodevelopmental studies suggest that the formation of the sympathetic nervous system requires intact NRG-1β, ErbB2, and ErbB3 signaling pathways. Targeted disruption of NRG-1β, ErbB2, or ErbB4 results in embryonic lethality due to defects in cardiac development. Recent studies have also highlighted the critical roles of NRG-1β, ErbB2, and ErbB4 in the cardiovascular development and maintenance of normal cardiac function in adults. Studies indicate that NRG-1β can enhance the sarcomere organizational structure of adult cardiomyocytes.

As used herein, “neuregulin” or “NRG” refers to a protein or peptide that bind to and activate ErbB2, ErbB3, ErbB4, or their heterologous or homodimers, including isoforms of neuregulin, EGF-like functional domains in neuregulin, polypeptides containing EGF-like functional domains of neuregulin, mutants or derivatives of neuregulin, and other neuregulin-like gene products that can activate the above receptors. Neuregulin also includes NRG-1, NRG-2, NRG-3, and NRG-4 proteins, peptides, fragments, and complexes with NRG-like function. Preferably, the neuregulin is a protein or polypeptide that can bind to and activate ErbB2/ErbB4 or ErbB2/ErbB3 heterodimers. As an example, but not for the purpose of limitation, the neuregulin of the present invention is a fragment of NRG-1β2 isomer, that is, amino acid fragment at positions 177-237, which contains EGF-like function domain. The amino acid sequence of the fragment is: SHLVKCAEKEKTFCVNGGECFMVKDLSNPSRYLCKCPNEFTGDRCQNYVMASFYKAEEL YQ (SEQ ID NO: 1). The neuregulin used in the invention can activate the above receptors and regulate their biological functions, such as stimulating skeletal muscle cells to synthesize acetylcholine receptors; promoting the differentiation, survival and DNA synthesis of myocardial cells. Neuregulin also includes mutants with conservative mutations that do not substantially affect biological functions, such as single amino acid mutation in non-critical regions (see Watson et al., Molecular Biology of the Gene, 4th Edition, 1987, The Benjamin/Cummings Pub. Co., P.224). Neuregulin used in the present invention can be isolated from natural sources, or obtained by recombinant technology, artificial synthesis or other means.

As used herein, “epidermal growth factor-like functional domain” or “EGF-like similar domain” or “EGF-like functional domain” refers to a polypeptide fragment encoded by neuregulin gene that can bind and activate ErbB2, ErbB3, ErbB4 or their heterologous or homodimer, and has structural similarity with EGF receptor binding regions described in the following references: Holmes et al., Science, 256: 1205-1210 (1992); U.S. Pat. Nos. 5,530,109 and 5,716,930; Hijazi et al., int.j.oncol., 13: 1061-1067 (1998); Chang et al., Nature, 387: 509-512 (1997); Carraway et al., Nature, 387: 512-516 (1997); Higashiyama et al., J. Biochem., 122: 675-680 (1997); and WO 97/09425. In certain embodiments, EGF-like functional domain binds to and activates ErbB2/ErbB4 or ErbB2/ErbB3 heterodimers. In some embodiments, the EGF-like functional domain comprises amino acids in the receptor binding region of NRG-1. In some embodiments, the EGF-like functional domain refers to amino acids at positions 177-226, 177-237 or 177-240 of NRG-1. In some embodiments, the EGF-like functional domain comprises amino acids in the receptor binding region of NRG-2. In some embodiments, the EGF-like functional domain comprises amino acids in the receptor binding region of NRG-3. In some embodiments, the EGF-like functional domain comprises amino acids in the receptor binding region of NRG-4. In certain embodiments, the EGF-like functional domain comprises the amino acid sequence described in U.S. Pat. No. 5,834,229: Ala Glu Lys Glu Lys Thr Phe Cys Val Asn Gly Gly Glu Cys Phe Met Val Lys Asp Leu Ser Asn Pro (SEQ ID NO: 2).

As used herein, “treatment” refers to any mode that can improve or change the symptoms of discomfort, disorder or disease in a better direction. Its effect can be preventive, completely or partially preventing a disease or symptoms thereof, or therapeutic, partially or completely curing a disease and/or adverse effects thereof. The treatment also includes any pharmaceutical use of the composition described herein.

As used herein, “heart failure” refers to an abnormality of heart function. In this case, the heart cannot supply blood according to the needs from tissue metabolism. Heart failure includes many diseases such as congestive heart failure, myocardial infarction, arrhythmia, familial hypertrophic cardiomyopathy, ischemic heart disease, idiopathic cardiomyopathy and myocarditis.

Heart failure can be caused by many factors, including but not limited to ischemic, congenital, rheumatic, viral, toxic or idiopathic heart failure. Chronic cardiac hypertrophy is a significant disease state in the early stage of congestive heart failure, which can cause cardiac arrest.

Unless the context specifies otherwise, the term ‘protein’ as used herein has the same meaning as “polypeptide” and “peptide segment”.

Unless the context specifies otherwise, the term “no more than” means “less than or equal to”.

Unless the context specifies otherwise, the term “about” as used herein means a value within 10% of a mentioned value.

As used herein, “LVESVI” refers to the left ventricular end-systolic volume index. As used herein, “LVEDVI” refers to the left ventricular end-diastolic volume index.

The left ventricular end-systolic volume index (LVESVI) is the volume/body surface area when the left ventricle contracts to the minimum, so that the evaluation of left ventricular end-systolic volume will not be affected by factors such as height and weight difference. LVESVI can be measured by two-dimensional (2D ECHO) and three-dimensional (3D ECHO) echocardiography, nuclear imaging, cardiac computed tomography and cardiac magnetic resonance imaging (CMR). In a healthy individual, LVESVI is usually in the range of 8 to 27 ml/m² for women and 9 to 31 ml/m² for men.

Left ventricular end-diastolic volume index (LVEDVI) is the end-diastolic volume/body surface area of left ventricle. LVEDVI can be measured by two-dimensional (2D ECHO) and three-dimensional (3D ECHO) echocardiography, nuclear imaging, cardiac computed tomography and cardiac magnetic resonance imaging (CMR). In a healthy individual, the LVEDVI of women is usually in the range of 29 to 70 ml/m², and usually in the range of 30 to 79 ml/m² for man.

Cardiac systolic function is related to factors such as preload, afterload, contractility and myocardial weight. LVESVI is an index to judge systolic function and prognosis. It is a clear and definite evidence that LVEDVI and LVESVI decrease at the same time in the same body, indicating that the patient's myocardial contractility is enhanced, cardiac function is improved, and ventricular remodeling is reversed, which can effectively reduce the mortality of patients with heart failure.

As used herein, “N-terminal brain natriuretic peptide” or “NT-proBNP” refers to the inactive precursor fragment of N-terminal brain natriuretic peptide (BNP). pro-BNP is the precursor of BNP, and BNP is an active natriuretic peptide related to hormones, which is mainly released by myocardial cells in the left ventricular wall. Prohormone proBNP is cleaved into BNP and inactive NT-proBNP by proteolysis under the stretching effect of myocardial wall.

The levels of BNP and NT-proBNP in plasma can be used to screen and diagnose heart failure, and are very useful for establishing the prognosis of heart failure, since these two markers are typically elevated in a patient with worsening condition. Furthermore, the method of the present invention includes using the BNP or NT-proBNP level to indicate a patient suitable for treating heart failure with neuregulin.

“New York Heart Association” or “NYHA” cardiac function classification used herein is a method to classify the degree of heart failure. It divides patients into four categories according to the degree of limitation on their physical activity ability. A limitation or symptom refers to normal breathing and different degrees of dyspnea and/or angina pectoris: I, asymptomatic and ordinary activities are not limited, such as dyspnea when walking or climbing stairs; II, Mild symptoms (mild dyspnea and/or angina pectoris) and mild restrictions on ordinary activities; III, Physical activity is obviously limited, even in the case of less than normal activity, for example, walking for a short distance (20-100 m), comfortable at rest; and IV, severely limit on the amount of activity, even when resting, there are symptoms, and most patients are bedridden.

### Advantages of the Invention

The neuregulin of the invention can improve the cardiac function of a patient with heart failure, especially improve LVESVI and LVEDVI of a single patient with heart failure at the same time. And, the inventors unexpectedly found that the neuregulin can improve the cardiac function of a patient with heart failure with high baseline LVESVI value, especially a patient with heart failure with LVESVI ≥135 m/m², and its effect is better than that of all patients with heart failure.

The effect of the neuregulin of the invention on improving the cardiac function of a patient with heart failure can be maintained for a long time, for example, until 90 days after administration.

The neuregulin of the invention directly acts on myocardial cells to repair their damaged structures and functions, enhance myocardial contractility, and reverse ventricular remodeling.

The technical solutions of the present invention will be further described below in connection with specific embodiments, however, following embodiments do not constitute a limitation of the present invention, and all the various embodiments based on the principles and technical means of the present invention shall fall within the scope of the present invention. Experimental methods for which specific conditions are not indicated in the following embodiments are generally in accordance with conventional conditions, or in accordance with conditions recommended by the manufacturer. Percentages and portions are calculated by weight unless otherwise indicated.

### Example 1: A Multi-Center, Randomized, Double-Blind, Placebo-Controlled and Standard Treatment-Based Phase III Clinical Trial to Evaluate the Effect of Recombinant Human Neuregulin for Injection on Cardiac Function of Subjects with Chronic Systolic Heart Failure

In this example, a multi-center, randomized, double-blind, placebo-controlled study was conducted based on standard treatment to evaluate the effects of recombinant human neuregulin for injection on the cardiac function of male patients with NT-proBNP ≤1700 pg/ml and female patients with NT-proBNP ≤4000 pg/ml, and to confirm its efficacy and safety.

### 1. Experimental Design:

A multicenter, randomized, double-blind, placebo-controlled study was conducted based on standard treatment.

Experimental group: recombinant human neuregulin for injection+standard basic treatment drugs (β receptor blocker, ACEI/ARB/ARNI, aldosterone receptor antagonist, etc.).

Placebo group: placebo+standard basic treatment drugs (0-blockers, ACEI/ARB/ARNI, aldosterone receptor antagonists, etc.).

### 2. Main Selection Criteria

2.1 Age is 18-75 years old, regardless of gender;
2.2 heart failure has been definitely diagnosed for 3 months or more, and the condition is stable at present, with NYHA II-III grade and left ventricular ejection fraction (LVEF)≤40%;
2.3 male Nt-proBNP ≤1700 pg/ml, female Nt-proBNP ≤4000 pg/ml;
2.4 After receiving standard basic treatment drugs for heart failure for 3 months, the target dose or maximum tolerated dose has been reached for more than 1 month, or the dose has not been changed within 1 month;
2.5 Understand and sign the informed consent form.

### 3. Test Drug:

### 3.1 Test Drug Name: Recombinant Human Neuregulin for Injection

Specification: Neucardin, 61 amino acids constitute EGF-like functional domain of Neuegulin-1β2 isomer, with molecular weight of 7054 Dal (1 μg=0.14 nmol); 250 μg/piece.
Dosage Form: Powder Injection
Administration Route: Intravenous Drip
Appearance: freeze-dried block or powder.

### 3.2 Placebo

Name: excipient of recombinant human neuregulin lyophilized powder for injection Specification: excipient of Neucardin (250 pg/piece, without active ingredients of recombinant human neuregulin)
Dosage form: powder injection
Administration route: intravenous drip
Appearance: lyophilized block or powder
Color: white or white-like.

### 4. The Administration Route, Dosage and Course of Treatment are Shown in Table 1:

TABLE 1
administration route, dosage and course of treatment

| | | |
|---|---|---|
| Dosage | 0 μg/kg/day (Placebo) | 0.6 μg/kg/day |

| | |
|---|---|
| Administration | Intravenous drip |

route

| | |
|---|---|
| Dosage | 50 ml |
| Course of | 10 hours a day for 10 consecutive days. |

treatment

5. Data Collection: Evaluating the Effectiveness and Safety During the Screening Period, 30 Days and 90 Days after Administration.

### 6. Results:

This example was carried out in many clinical research centers in China at the same time. 154 cases were actually enrolled, 78 cases in the Neuregulin experimental group and 76 cases in the placebo group. A total of 147 cases (73 cases in the experimental group and 74 cases in the placebo group) finally achieved the main end points.

### 6.1 LVESVI

According to the obtained 30-day data, 63% of the patients in the Neuregulin experimental group (see FIG. 1) had LVESVI reduction, and the proportion of patients with LVESVI reduction at 30 days compared with the baseline was also higher than that in the placebo group.

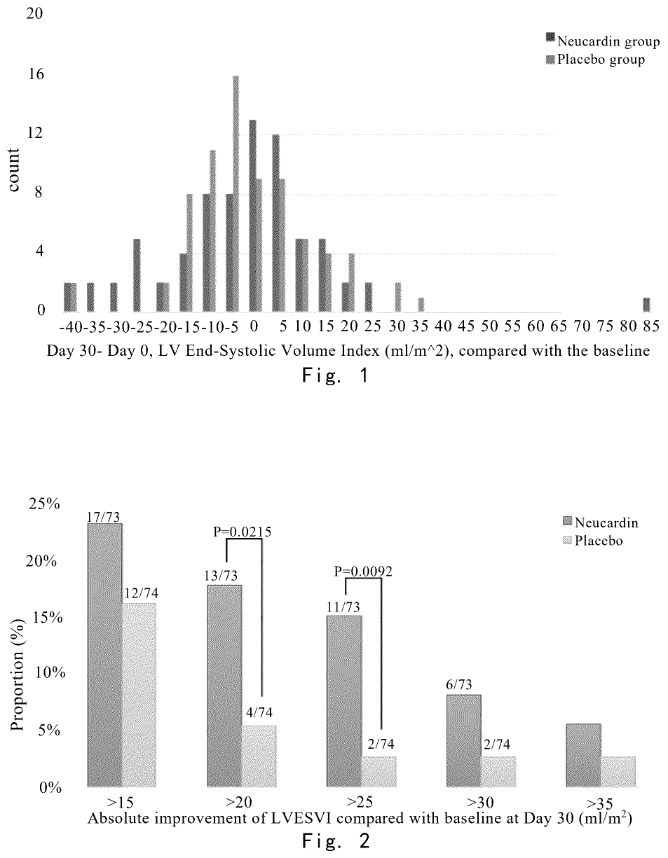

Improvement value of LVESVI was stratified compared with the baseline (greater than or equal to 5, 10, 15, 20, 25, 30, 35, 40 ml/m²) 30 days after administration, and the improvement rates of Neuregulin group and placebo group at each level were calculated respectively. LVESVI was decreased by ≥5, 10, 15, 20, 25, 30, 35 and 40 ml/m² compared with the baseline, the corresponding improvement rates in Neuregulin group were 45.2%, 34.2%, 23.3%, 17.8%, 15.1%, 8.2%, 5.5% and 2.7% respectively, and the corresponding improvement rates in placebo group were 52.7%, 31.1%, 16.2%, 5.4%, 2.7%, 2.7%, 2.7% and 2.7% respectively. Compared with the placebo group, with the increasing improvement, there was a significant difference between the number of people in Neuregulin group and the placebo group, suggesting that the improvement effects of Neuregulin on LVESVI was better than that of the placebo group. When the improvement on LVESVI was greater than or equal to 20 ml/m² and 25 ml/m², the improvement rate between the two groups was statistically significant (P<0.05, see FIG. 2).

In addition, by analyzing the results of two CMR tests during the stable period within one month before the subjects entered the group, it was found that, under the existing standard treatment conditions, the natural occurrence ratio of patients with LVESVI decreased by more than ml/m² within one month without the intervention of the test drug was 2.58%, which was close to the ratio of patients with LVESVI improved by more than 25 ml/m² within one month after the administration drug in the placebo group (2.7%). It shows that the improvement rate of LVESVI more than 25 ml/m² within one month is stable, while the improvement rate of neuregulin group achieving such improvement within one month was 15.1%, which was significantly higher than the natural level. Summing up, it shows that the drug can significantly improve the rate of cardiac function improvement of the subjects.

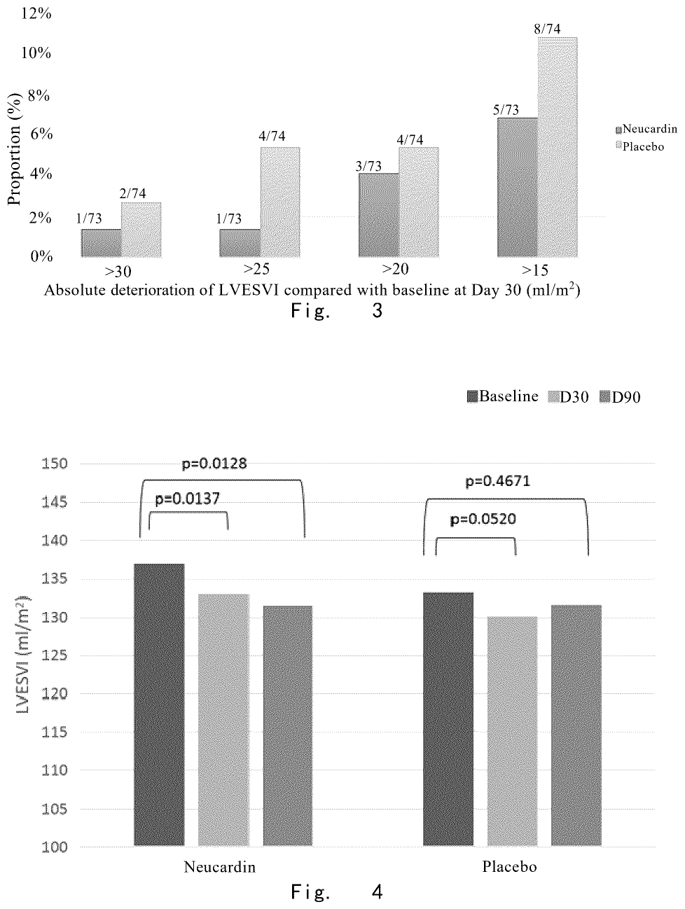

However, through the analysis of the difference in the deterioration of cardiac function between the Neuregulin experimental group and the placebo group for 30 days (see Table 2 and FIG. 3), the proportion of LVESVI increase in the Neuregulin experimental group was lower than that in the placebo group, which shows a consistent trend with the improvement of cardiac function by the decrease of LVESVI, indicating that Neuregulin can prevent the deterioration of cardiac function in the subjects.

TABLE 2
LVESVI increased by 5, 10, 15, 20, 25 and 30
ml/m² at Day 30 compared with the baseline (FAS)

| | n/N(%) | |
|---|---|---|
| | Experimental | Placebo |
| Variable | group | group |
| Increase in LVESVI at Day 30 compared | 15/73(20.5) | 17/74(23.0) |
| with the baseline was more than or | | |
| equal to 5 ml/m² | | |
| Increase in LVESVI at Day 30 compared | 10/73(13.7) | 12/74(16.2) |
| with the baseline was more than or | | |
| equal to 10 ml/m² | | |
| Increase in LVESVI at Day 30 compared | 5/73(6.8) | 8/74(10.8) |
| with the baseline was more than or | | |
| equal to 15 ml/m² | | |
| Increase in LVESVI at Day 30 compared | 3/73(4.1) | 4/74(5.4) |
| with the baseline was more than or | | |
| equal to 20 ml/m² | | |
| Increase in LVESVI at Day 30 compared | 1/73(1.4) | 4/74(5.4) |
| with the baseline was more than or | | |
| equal to 25 ml/m² | | |
| Increase in LVESVI at Day 30 compared | 1/73(1.4) | 2/74(2.7) |
| with the baseline was more than or | | |
| equal to 30 ml/m² | | |

NOTE:
N = Number of subjects whose LVESVI index was obtained in the group;
n = Number of specific categories

The cardiac function in Neuregulin group was measured at Day 30 after administration. Compared with that before administration, LVESVI was improved by 5.62 ml/m², which was statistically significant (P=0.0137), and the improvement effect could be maintained until Day 90 after the administration (P=0.0128). However, there was no statistically significant difference in cardiac function in the placebo group at Day 30 and 90 after the administration (P ≥0.05) (see Table 3 and FIG. 4).

TABLE 3
Day 30, 90 ΔLVESVI

| ΔLVESVI | NRG experimental group | Placebo group |
|---|---|---|
| 30 D | −5.62 ± 18.99 | −3.31 ± 14.43 |
| 90 D | −5.23 ± 17.12 | −1.51 ± 17.28 |

### 6.2 LVEDVI

According to the obtained 30-day LVEDVI data, the proportion of patients with LVEDVI reduction at 30 days compared with the baseline was higher than that in the placebo group. At Day 30 after the administration, LVEDVI was decreased by ≥5, 10, 15, 20, 25, 30, 35 and 40 ml/m² compared with the baseline, the corresponding improvement rates in Neuregulin group were 43.8%, 30.1%, 21.9%, 17.8%, 16.4%, 13.7%, 6.8% and 5.5% respectively, and the corresponding improvement rates in placebo group were 44.6%, 29.7%, 14.9%, 12.2%, 6.8%, 4.1%, 4.1% and 2.7% respectively.

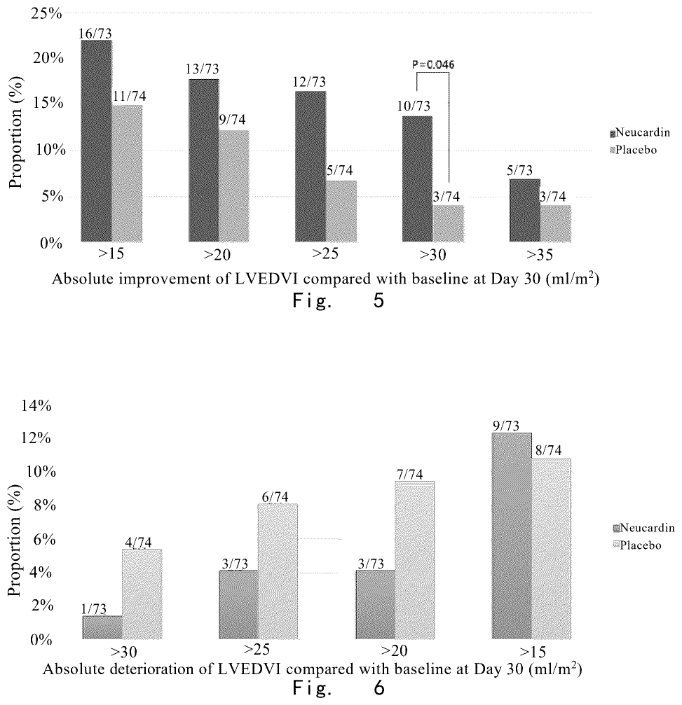

Especially, when the decrease of LVEDVI is equal to or greater than 30 ml/m², there are 10 people in the Neuregulin experimental group, with an improvement rate of 13.7%, and 3 people in the placebo group, with an improvement rate of 4.1% (P value of 0.046, see Table 4 and FIG. 5). The proportion of people in the Neuregulin experimental group is significantly higher than that in the placebo group, and the difference is statistically significant. It shows that the drug can significantly improve the improvement rate of cardiac function of the subjects.

TABLE 4
LVEDVI reduced by 5, 10, 15, 20, 25, 30, 35 and 40
ml/m² at Day 30 compared with the baseline (FAS)

| | n/N(%) | |
|---|---|---|
| | Experimental | Placebo |
| Variable | group | group |
| Reduction in LVEDVI at Day 30 compared | 32/73(43.8) | 33/74(44.6) |
| with the baseline was more than or | | |
| equal to 5 ml/m² | | |
| Reduction in LVEDVI at Day 30 compared | 22/73(30.1) | 22/74(29.7) |
| with the baseline was more than or | | |
| equal to 10 ml/m² | | |
| Reduction in LVEDVI at Day 30 compared | 16/73(21.9) | 11/74(14.9) |
| with the baseline was more than or | | |
| equal to 15 ml/m² | | |
| Reduction in LVEDVI at Day 30 compared | 13/73(17.8) | 9/74(12.2) |
| with the baseline was more than or | | |
| equal to 20 ml/m² | | |
| Reduction in LVEDVI at Day 30 compared | 12/73(16.4) | 5/74(6.8) |
| with the baseline was more than or | | |
| equal to 25 ml/m² | | |
| Reduction in LVEDVI at Day 30 compared | 10/73(13.7) | 3/74(4.1) |
| with the baseline was more than or | | |
| equal to 30 ml/m² | | |
| Reduction in LVEDVI at Day 30 compared | 5/73(6.8) | 3/74(4.1) |
| with the baseline was more than or | | |
| equal to 35 ml/m² | | |
| Reduction in LVEDVI at Day 30 compared | 4/73(5.5) | 2/74(2.7) |
| with the baseline was more than or | | |
| equal to 40 ml/m² | | |

NOTE:
N = Number of subjects whose LVESVI index was obtained in the group;
n = Number of specific categories

The analysis of the difference in the deterioration of cardiac function between the neuregulin group and the placebo group over 30 days (as shown in Table 5 and FIG. 6) revealed that the proportion of patients with increased LVEDVI was lower in the neuregulin group compared with the placebo group. This finding is consistent with the previous analysis that showed a reduction in LVEDVI leading to improved cardiac function, indicating that neuregulin can prevent the worsening of cardiac function in patients.

TABLE 5
LVEDVI increased by 5, 10, 15, 20, 25 and 30
ml/m² at Day 30 compared with the baseline (FAS)

| | n/N(%) | |
|---|---|---|
| | Experimental | Placebo |
| Variable | group | group |
| Increase in LVEDVI at Day 30 compared | 19/73(26.0) | 18/74(24.3) |
| with the baseline was more than or | | |
| equal to 5 ml/m² | | |
| Increase in LVEDVI at Day 30 compared | 14/73(19.2) | 13/74(17.6) |
| with the baseline was more than or | | |
| equal to 10 ml/m² | | |
| Increase in LVEDVI at Day 30 compared | 9/73(12.3) | 8/74(10.8) |
| with the baseline was more than or | | |
| equal to 15 ml/m² | | |
| Increase in LVEDVI at Day 30 compared | 3/73(4.1) | 7/74(9.5) |
| with the baseline was more than or | | |
| equal to 20 ml/m² | | |
| Increase in LVEDVI at Day 30 compared | 3/73(4.1) | 6/74(8.1) |
| with the baseline was more than or | | |
| equal to 25 ml/m² | | |
| Increase in LVEDVI at Day 30 compared | 1/73(1.4) | 4/74(5.4) |
| with the baseline was more than or | | |
| equal to 30 ml/m² | | |

NOTE:
N = Number of subjects whose LVESVI index was obtained in the group;
n = Number of specific categories

### 6.3 LVESVI and LVEDVI

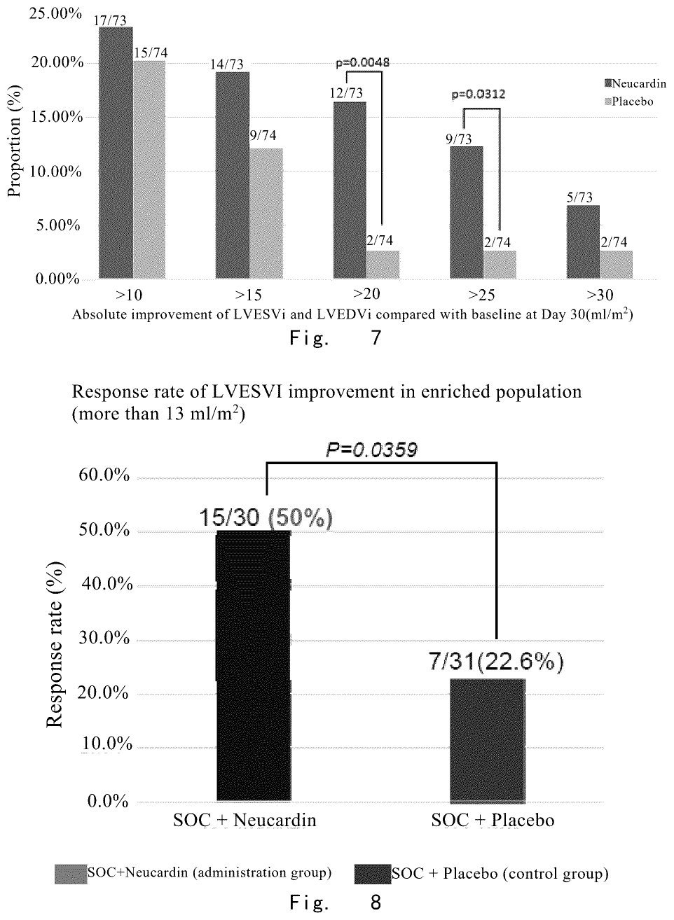

Based on the LVESVI and LVEDVI data obtained from the individual subjects, the LVESVI and LVEDVI of an individual subject was decreased by ≥5, 10, 15, 20, 25, 30, 35 and 40 ml/m² compared with the baseline at Day 30 after the administration. The corresponding improvement rates in the Neuregulin group were 37.0%, 23.3%, 19.2%, 16.4%, 12.3%, 6.8%, 4.1%, and 1.4%, respectively. In the placebo group, the corresponding improvement rates were 39.2%, 20.3%, 12.2%, 2.7%, 2.7%, 2.7%, 2.7%, and 1.4%. The LVESV and LVEDV of the individual subjects improved simultaneously. Especially, when LVESVI and LVEDV were decreased by 20 ml/m², in the Neuregulin group, 12 subjects showed improvement with an improvement rate of 16.4%, while in the placebo group, 2 subjects showed improvement with an improvement rate of 2.7%. The p-value was 0.0048 (see Table 6 and FIG. 7), indicating a statistically significant difference. This suggests that Neuregulin can improve cardiac function and significantly reverse ventricular remodeling.

TABLE 6
LVESVI and LVEDVI decreased by 5, 10, 15, 20,
25, 30, 35 and 40 ml/m² in an individual subject
at Day 30 compared with the baseline (FAS)

n/N(%)

| | Experimental | Placebo |
|---|---|---|
| Variable (LVESVI and LVEDVI) | group | group |
| Simultaneous reduction by more | 27/73(37.0) | 29/74(39.2) |
| than or equal to 5 ml/m² | | |
| Simultaneous reduction by more | 17/73(23.3) | 15/74(20.3) |
| than or equal to 10 ml/m² | | |
| Simultaneous reduction by more | 14/73(19.2) | 9/74(12.2) |
| than or equal to 15 ml/m² | | |
| Simultaneous reduction by more | 12/73(16.4) | 2/74(2.7) |
| than or equal to 20 ml/m² | | |
| Simultaneous reduction by more | 9/73(12.3) | 2/74(2.7) |
| than or equal to 25 ml/m² | | |
| Simultaneous reduction by more | 5/73(6.8) | 2/74(2.7) |
| than or equal to 30 ml/m² | | |
| Simultaneous reduction by more | 3/73(4.1) | 2/74(2.7) |
| than or equal to 35 ml/m² | | |
| Simultaneous reduction by more | 1/73(1.4) | 1/74(1.4) |
| than or equal to 40 ml/m² | | |

NOTE:
N = Number of subjects whose LVESVI index was obtained in the group;
n = Number of specific categories

The analysis of the difference in the deterioration of cardiac function between the neuregulin group and the placebo group over 30 days (as shown in Table 7) revealed that the proportion of individual subjects with increased LVESVI and LVEDVI was lower in the neuregulin group compared with the placebo group. This finding is consistent with the previous analysis that showed a reduction in both LVEDSI and LVEDVI leading to improved cardiac function, indicating that neuregulin can prevent the worsening of cardiac function in patients.

TABLE 7
Both LVESVI and LVEDVI increased by 5, 10, 15, 20, 25
and 30 ml/m² at Day 30 compared with the baseline (FAS)

| | n/N(%) | |
|---|---|---|
| | Experimental | Placebo |
| Variable (LVESVI and LVEDVI) | group | group |
| Simultaneous increase by more than | 13/73(17.8) | 11/74(14.9) |
| or equal to 5 ml/m² | | |
| Simultaneous increase by more than | 9/73(12.3) | 10/74(13.5) |
| or equal to 10 ml/m² | | |
| Simultaneous increase by more than | 3/73(4.1) | 7/74(9.5) |
| or equal to 15 ml/m² | | |
| Simultaneous increase by more than | 2/73(2.7) | 4/74(5.4) |
| or equal to 20 ml/m² | | |
| Simultaneous increase by more than | 1/73(1.4) | 4/74(5.4) |
| or equal to 25 ml/m² | | |
| Simultaneous increase by more than | 1/73(1.4) | 2/74(2.7) |
| or equal to 30 ml/m² | | |

NOTE:
N = Number of subjects whose LVESVI index was obtained in the group;
n = Number of specific categories

### Example 2. Study on Improving Effects of Cardiac Function in Enriched Subgroup Populations—1

Through statistical analysis of the experimental results from Example 1, the inventors investigated, in this Example, the response rate of Neuregulin in improving LVESVI (by more than 13 mL/m²) in an enriched population. The enriched population refers to patients with a baseline LVESVI ≥135 mL/m², accounting for approximately 41% of the total studied population. These patients generally suffer from severe heart failure and exhibit significant ventricular remodeling.

The results unexpectedly revealed (as shown in FIG. 8) that Neuregulin demonstrated a better improvement in cardiac function in the enriched subgroup (baseline LVESVI ≥135 mL/m², representing about 41% of the studied population).

The response rate for a substantial improvement in LVESVI (a reduction of more than 13 mL/m²) in the Neuregulin treatment group reached 50%, significantly higher than that in the placebo group.

The above results indicate that the larger the baseline LVESVI, the larger the heart, and the better the therapeutic effects of Neuregulin on larger hearts.

### Example 3. Study on Improving Effects of Cardiac Function in Enriched Subgroup Populations—2

Through statistical analysis of the results from Example 1, the inventors investigated, in this Example, the response rate of Neuregulin in improving both LVESVI and LVEDVI (both exceeding 20 mL/m²) in individual patients of the enriched population.

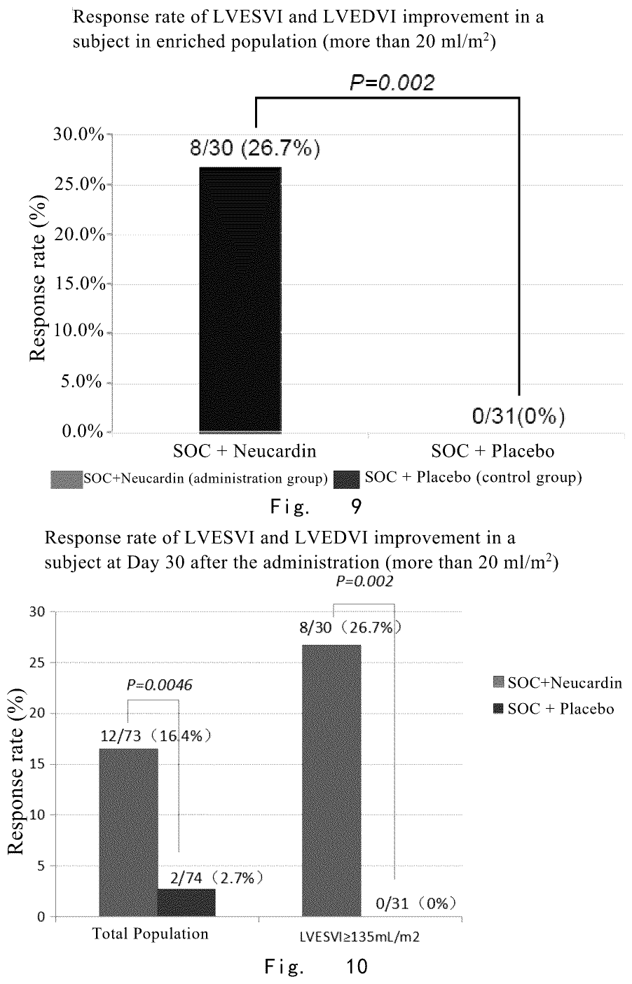

The results unexpectedly revealed (as shown in FIG. 9) that: in the enriched population (baseline LVESVI ≥135 mL/m²), the response rate of patients in the Neuregulin treatment group experiencing significant improvements in both LVESVI and LVEDVI (both indices decreased by more than 20 mL/m²) was significantly higher than that in the placebo group.

At the same time, by comparing the response rates of patients in both the general population and the enriched population (baseline LVESVI ≥135 mL/m²) who received either Neuregulin or placebo, and who experienced significant improvements in both LVESVI and LVEDVI (both indicators decreased by more than 20 mL/m²), the present invention unexpectedly found that although neuregulin treatment group in both the general and enriched populations showed significantly higher rates of significant improvement in both LVESVI and LVEDVI compared with the placebo group, the neuregulin treatment group in the enriched population exhibited a better response rate for significant improvements in both LVESVI and LVEDVI (both indicators decreased by more than 20 mL/m²) than the neuregulin treatment group in the general population, as shown in FIG. 10.

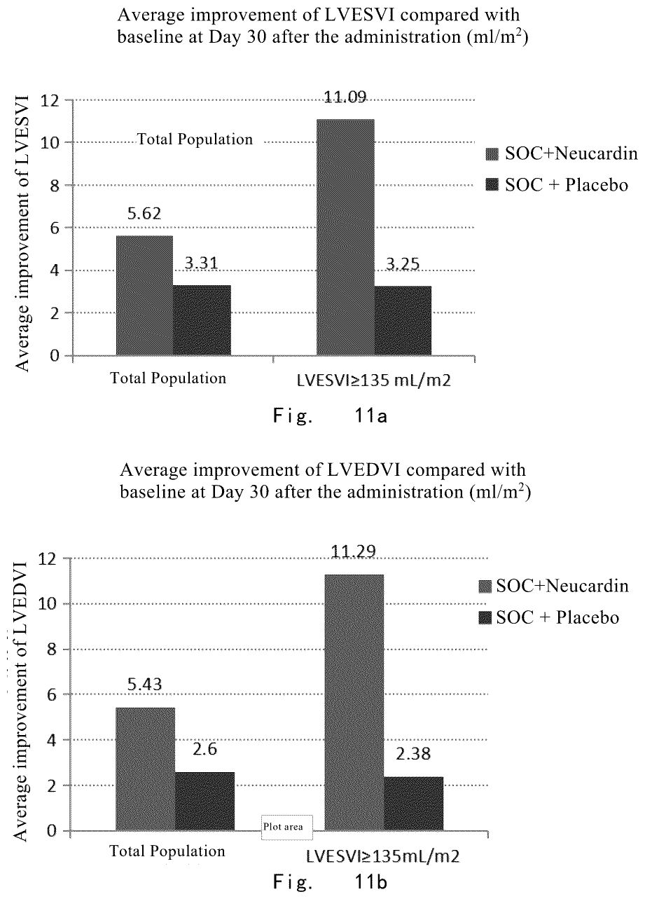

The present inventors further analyzed the average improvements in LVESVI or LVEDVI compared with baseline at Day 30 after the administration of neuregulin or placebo in both the general and enriched populations. It was also unexpectedly found that although the neuregulin treatment group in both the general and enriched populations showed better average improvements in LVESVI or LVEDVI compared with the placebo group, the neuregulin treatment group in the enriched population demonstrated better average improvements in LVESVI or LVEDVI than the neuregulin treatment group in the general population, as shown in FIGS. 11a and 11b. These results suggest that neuregulin has a more pronounced effect on improving cardiac function in this enriched population, thereby being more effective in preventing, treating heart failure, or delaying the onset of heart failure in this specific group.

Therefore, neuregulin demonstrates better efficacy in the enriched population (baseline LVESVI ≥135 mL/m²): the response rate for an improvement in LVESVI greater than 13 mL/m² in the treatment group was 50%, which is statistically significant; the response rate for individual subjects showing simultaneous improvements in both LVESVI and LVEDVI greater than 20 mL/m² was 26.5% (compared with 0% in the placebo group, which is statistically significant).

All documents mentioned herein are cited as references in the present application as if each document is cited individually as a reference. It is further to be understood that after reading the foregoing teachings of the present invention, a skilled person can make various alterations or modifications to the present invention, and such equivalent forms will likewise fall within the scope of the claims appended to the present application.

1. Use of a neuregulin or a functional fragment thereof, or a nucleic acid encoding the neuregulin or a functional fragment thereof, or a substance that enhances the production and/or function of the neuregulin in the human body, in the preparation of a drug for preventing, treating a patient with heart failure, or delaying the onset of heart failure in a patient, and the drug can improve cardiac function in a patient with heart failure.

2. Use of the neuregulin in the preparation of a drug for preventing, treating a patient with heart failure, delaying the onset of heart failure in a patient, or improving cardiac function in a patient with heart failure.

3. The use of claim 1, wherein the patient with severe heart failure represents a population with severe ventricular remodeling.

4. The use of claim 1, wherein the patient with heart failure has an LVESVI of ≥100 mL/m², ≥110 mL/m², ≥120 mL/m², ≥130 mL/m², ≥135 mL/m², ≥140 mL/m², ≥145 mL/m², ≥150 mL/m², ≥155 mL/m², ≥160 mL/m², ≥165 mL/m², or ≥170 mL/m² at baseline before administration; preferably ≥135 mL/m²; or
the patient with heart failure has an LVESVI of 100-350 mL/m², 110-350 mL/m², 120-350 mL/m², 130-350 mL/m², or 135-350 mL/m² at baseline before administration; preferably 135-350 mL/m².

5. The use of claim 1, wherein the patient with heart failure is a patient with chronic heart failure.

6. The use of claim 1, wherein the patient with heart failure is classified as NYHA II or III according to the New York Heart Association (NYHA) functional classification.

7. The use of claim 1, wherein the patient with heart failure has a plasma NT-proBNP level not exceeding 4000 pg/ml.

8. The use of claim 1, wherein, in the patient with heart failure, a male patient has a plasma NT-proBNP level not exceeding 1700 pg/ml, and a female patient has a plasma NT-proBNP level not exceeding 4000 pg/ml.

9. The use of claim 1, wherein “improving cardiac function in the patient with heart failure” refers to improving LVESVI and/or LVEDVI.

10. The use of claim 1, wherein, after the drug is given, the LVESVI of the patient with heart failure is reduced by an amount equal to or greater than 10 mL/m²; preferably equal to or greater than 20 mL/m².

11. The use of claim 1, wherein, after the drug is given, the LVEDVI of the patient with heart failure is reduced by an amount equal to or greater than 10 mL/m²; preferably equal to or greater than 20 mL/m².

12. The use of claim 1, wherein “improving cardiac function in the patient with heart failure” refers to simultaneously improving both LVESVI and LVEDVI in an individual patient with heart failure.

13. The use of claim 12, wherein both LVESVI and LVEDVI are simultaneously reduced by an amount equal to or greater than 20 or 25 mL/m² in an individual patient with heart failure.

14. The use of claim 1, wherein the patient with heart failure experience reversal of ventricular remodeling.

15. The use of claim 1, wherein the neuregulin comprises neuregulin 1 (NRG-1), neuregulin 2 (NRG-2), neuregulin 3 (NRG-3), and neuregulin 4 (NRG-4).

16. The use of claim 1, wherein the neuregulin comprises neuregulin 1 (NRG-1) or the EGF-like domain of neuregulin 1 (NRG-1).

17. The use of claim 1, wherein the neuregulin is a protein, the amino acid sequence of which is shown in SEQ ID NO:1.

18. A method for preventing, treating a patient with heart failure, or delaying the onset of heart failure in a patient, comprising administering to a patient in need thereof an effective amount of neuregulin or a functional fragment thereof, or a nucleic acid encoding the neuregulin or a functional fragment thereof, or a substance that enhances the production and/or function of the neuregulin in the human body, and the method can improve cardiac function in the patients with heart failure.

19. A method for preventing, treating a patient with heart failure, delaying the onset of heart failure in a patient, or improving cardiac function in a patient with heart failure, comprising administering to a patient in need thereof an effective amount of neuregulin.

20. The use of claim 18, wherein the patient with heart failure represents a population with severe ventricular remodeling.

21. The method of claim 18, wherein the patient with heart failure has an LVESVI of ≥100 mL/m², ≥110 mL/m², ≥120 mL/m², ≥130 mL/m², ≥135 mL/m², ≥140 mL/m², ≥145 mL/m², ≥150 mL/m², ≥155 mL/m², ≥160 mL/m², ≥165 mL/m², or ≥170 mL/m² at baseline before administration; preferably ≥135 mL/m²; or
the patient with heart failure has an LVESVI of 100-350 mL/m², 110-350 mL/m², 120-350 mL/m², 130-350 mL/m², or 135-350 mL/m² at baseline before administration; preferably 135-350 mL/m².

22. The method of claim 18, wherein the patient with heart failure is a patient with chronic heart failure.

23. The method of claim 18, wherein the patient with heart failure is classified as NYHA II or III according to the New York Heart Association (NYHA) functional classification.

24. The method of claim 18, wherein the patient with heart failure has plasma NT-proBNP levels not exceeding 4000 pg/ml.

25. The method of claim 18, wherein, in the patient with heart failure, a male patient has plasma NT-proBNP levels not exceeding 1700 pg/ml, and a female patient has plasma NT-proBNP levels not exceeding 4000 pg/ml.

26. The method of claim 18, wherein “improving cardiac function in the patient with heart failure” refers to improving LVESVI and/or LVEDVI.

27. The method of claim 18, wherein, after the administration, the LVESVI of the patient with heart failure is reduced by an amount equal to or greater than 10 mL/m²; preferably equal to or greater than 20 mL/m².

28. The method of claim 18, wherein, after the administration, the LVEDVI of the patient with heart failure is reduced by an amount equal to or greater than 10 mL/m²; preferably equal to or greater than 20 mL/m².

29. The method of claim 18, wherein “improving cardiac function in the patient with heart failure” refers to simultaneously improving both LVESVI and LVEDVI in an individual patient with heart failure.

30. The method of claim 29, wherein both LVESVI and LVEDVI are simultaneously reduced by an amount equal to or greater than 20 or 25 mL/m² in an individual patient with heart failure.

31. The use of claim 18, wherein the patient with heart failure experience reversal of ventricular remodeling.

32. The method of claim 18, wherein the neuregulin comprises neuregulin 1 (NRG-1), neuregulin 2 (NRG-2), neuregulin 3 (NRG-3), and neuregulin 4 (NRG-4).

33. The method of claim 18, wherein the neuregulin comprises neuregulin 1 (NRG-1) or the EGF-like domain of neuregulin 1 (NRG-1).

34. The method of claim 18, wherein the neuregulin is a protein, the amino acid sequence of which is shown in SEQ ID NO:1.

35. The method of claim 18, wherein the method comprises administering neuregulin to a patient via an introduction regimen.

36. The method of claim 35, wherein the introduction regimen comprises continuously administering the drug component for at least 3, 5, 7, or 10 consecutive days.

37. The method of claim 35, wherein the method comprises administering a maintenance regimen to the patient for at least 3, 6, or 12 months.

38. The method of claim 37, wherein the maintenance regimen comprises administering the drug every 3, 5, 7, or 10 days.

39. The method of claim 18, wherein the method further comprises a step of assessing whether the patient in need of preventing, treating or delaying heart failure is suitable for neuregulin therapy prior to administering the treatment to the patient.

40. The method of claim 39, wherein the step of assessing whether the patient is suitable for neuregulin therapy comprises performing a NYHA cardiac function classification assessment on the patient with heart failure.

41. The method of claim 40, wherein a patient with NYHA Class II or NYHA Class III according to NYHA cardiac function classification is suitable for neuregulin therapy.

42. The method of claim 39, wherein the step of assessing whether the patient is suitable for neuregulin therapy further comprises measuring plasma NT-proBNP in the patient with heart failure.

43. The method of claim 42, wherein the plasma NT-proBNP level does not exceed 4000 pg/ml.

44. The method of claim 42, wherein a male patient with heart failure has a plasma NT-proBNP level not exceeding 1700 pg/ml, and a female patient has a plasma NT-proBNP level not exceeding 4000 pg/ml.
